(19) United States
(12) Patent Application Publication
(10) Pub. No.: US 2021/0000778 A1
(43) Pub. Date: Jan. 7, 2021

(54) THERAPEUTIC METHODS AND COMPOSITIONS FOR TREATING PROSTATE CANCER USING 6,8-BIS-BENZYLTHIO-OCTANOIC ACID

(71) Applicant: Rafael Pharmaceuticals, Inc., Cranbury, NJ (US)

(72) Inventors: Sanjeev Luther, Cranbury, NJ (US); Robert G.L. Shorr, Edison, NJ (US); Paul Bingham, South Setauket, NY (US); Zuzana Zachar, South Setauket, NY (US)

(21) Appl. No.: 16/980,885

(22) PCT Filed: Apr. 16, 2019
(86) PCT No.: PCT/US2019/027597

§ 371 Date: Sep. 15, 2020

Related U.S. Application Data
(60) Provisional application No. 62/782,938, filed on Dec. 20, 2018.
(60) Provisional application No. 62/658,238, filed on Apr. 16, 2018.

(51) Int. Cl. A61K 31/20; A61K 31/337; A61K 9/00; A61P 35/04

(52) U.S. Cl. CPC A61K 31/20; A61K 31/337; A61P 35/04; A61K 9/0053; A61K 9/0019

(57) ABSTRACT

The invention provides methods, compositions, and medical kits for treating prostate cancer using 6,8-bis-benzylthio-octanoic acid or a pharmaceutically acceptable salt thereof, including combination therapy with docetaxel.

### CROSS-REFERENCE TO RELATED APPLICATIONS

This application claims the benefit of and priority to U.S. Provisional Patent Application Ser. No. 62/658,238, filed Apr. 16, 2018, and U.S. Provisional Patent Application Ser. No. 62/782,938, filed Dec. 20, 2018; the contents of each of which are hereby incorporated by reference.

### FIELD OF THE INVENTION

The invention provides methods, compositions, and medical kits for treating prostate cancer using 6,8-bis-benzylthio-octanoic acid (CPI-613) or a pharmaceutically acceptable salt thereof, including combination therapy with docetaxel.

### BACKGROUND

Cancer is a leading cause of death in many industrialized countries. Recent estimates are that 10 million Americans are currently living with cancer, and that 1.2 million Americans are newly diagnosed with cancer each year. Significant advances have been made in improving the diagnosis and treatment of cancer. However, current treatment options often suffer from severe adverse side effects and/or the treatments are not effective for all patients. For example, many clinically-accepted chemotherapeutic agents can induce profound damage to normal, proliferative host cells. Another problem associated with many chemotherapeutic treatments is that, in many tumor types, there is either inherent or acquired resistance to the therapy.

Prostate cancer affects a substantial portion of the male patient population. Prostate cancer is characterized by the formation of malignant cells in the prostate. Exemplary therapies currently used to help treat patients suffering from prostate cancer include surgery, radiation therapy, and chemotherapy. Surgery often includes the complete surgical removal of the prostate gland. Radiation therapy involves applying ionizing radiation to the diseased area of the prostate. However, not all patients achieve sufficient remission of prostate cancer using existing therapies and/or experience adverse side effects when subjected to existing therapies.

Accordingly, the need exists for new therapeutic methods that provide improved efficacy and/or reduced side effects for treating prostate cancer. The present invention addresses this need and provides other related advantages.

### SUMMARY

The invention provides methods, compositions, and medical kits for treating prostate cancer using 6,8-bis-benzylthio-octanoic acid or a pharmaceutically acceptable salt thereof, including combination therapy with docetaxel. The prostate cancer may be, for example, characterized as an androgen-dependent prostate cancer or an androgen-independent prostate cancer. The 6,8-bis-benzylthio-octanoic acid or a pharmaceutically acceptable salt thereof may be formulated as a pharmaceutical composition, such as a pharmaceutical composition containing an ion pairing agent. When used as part of a combination therapy, the 6,8-bis-benzylthio-octanoic acid or a pharmaceutically acceptable salt thereof may be formulated as a pharmaceutical composition for administration to the patient separate from a pharmaceutical composition containing other agent(s) used in the combination therapy, such as docetaxel. The methods, compositions, and medical kits for treating prostate cancer provide particular benefits to adult, human male patients suffering from prostate cancer.

Accordingly, one aspect of the invention provides a method for treating a cancer selected from the group consisting of androgen-dependent prostate cancer and androgen-independent prostate cancer. The method comprises administering to a patient in need thereof a therapeutically effective amount of a first therapeutic agent comprising 6,8-bis-benzylthio-octanoic acid or a pharmaceutically acceptable salt thereof, in order to treat the cancer. The first therapeutic agent may be administered, for example, by oral or intravenous administration.

Another aspect of the invention provides a method for treating prostate cancer, where the method comprises administering to a patient in need thereof a therapeutically effective amount of (i) a first therapeutic agent comprising 6,8-bis-benzylthio-octanoic acid or a pharmaceutically acceptable salt thereof and (ii) a second therapeutic agent comprising docetaxel or a pharmaceutically acceptable salt thereof, in order to treat the prostate cancer. The method may be characterized according to, for example, the type of prostate cancer, such as an androgen-dependent prostate cancer or an androgen-independent prostate cancer. The first therapeutic agent may be administered, for example, by oral or intravenous administration.

Another aspect of the invention provides a method for treating prostate cancer according to a particular dosing amount. The method comprises administering to a patient in need thereof a unit dosage of a first therapeutic agent comprising 6,8-bis-benzylthio-octanoic acid or a pharmaceutically acceptable salt thereof in an amount ranging from about 150 mg/m² to about 1500 mg/m², to treat the prostate cancer. The method may be characterized according to, for example, the type of prostate cancer, such as an androgen-dependent prostate cancer or an androgen-independent prostate cancer. The first therapeutic agent may be administered, for example, by oral or intravenous administration.

Another aspect of the invention provides a medical kit for treating a cancer selected from the group consisting of androgen-dependent prostate cancer and androgen-independent prostate cancer. The kit comprises (i) a first therapeutic agent comprising 6,8-bis-benzylthio-octanoic acid or a pharmaceutically acceptable salt thereof, and (ii) instructions for treating a cancer selected from the group consisting of androgen-dependent prostate cancer and androgen-independent prostate cancer using said first therapeutic agent. The instructions may specify, for example, the route of administration for the first therapeutic agent, such as by oral or intravenous administration.

Another aspect of the invention provides a medical kit for treating prostate cancer. The kit comprises (i) a first therapeutic agent comprising 6,8-bis-benzylthio-octanoic acid or a pharmaceutically acceptable salt thereof, and (ii) instructions for treating prostate cancer using the first therapeutic agent in combination with a second therapeutic agent comprising docetaxel or a pharmaceutically acceptable salt thereof. The instructions may specify, for example, the type of prostate cancer to be treated, such as an androgen-dependent prostate cancer or an androgen-independent prostate cancer. The instructions may specify, for example, the route of administration for the first therapeutic agent, such as by oral or intravenous administration.

Another aspect of the invention provides a medical kit for treating prostate cancer according to particular dosing amount. The kit comprises (i) a first therapeutic agent comprising 6,8-bis-benzylthio-octanoic acid or a pharmaceutically acceptable salt thereof, and (ii) instructions for treating prostate cancer using a dosage of the first therapeutic agent ranging from about 150 mg/m² to about 1500 mg/m². The instructions may specify, for example, the type of prostate cancer to be treated, such as an androgen-dependent prostate cancer or an androgen-independent prostate cancer. The instructions may specify, for example, the route of administration for the first therapeutic agent, such as by oral or intravenous administration.

The foregoing aspects of the invention are described in more detail, along with additional embodiments, in the detailed description below.

### BRIEF DESCRIPTION OF FIGURES

FIG. 1 is graph a showing the percentage of remaining live PC-3 androgen resistant prostate cancer cells observed following the treatment protocol, as described in Example 2.

FIG. 2 is graph showing observed tumor volume over time after administration of 2.5 mg/kg or 10 mg/kg CPI-613 according to the procedures described in Example 3.

FIG. 3 is graph showing mouse survival time after administration of 2.5 mg/kg or 10 mg/kg CPI-613 according to the procedures described in Example 3.

FIG. 4 depicts the anti-tumor efficacy of oral 6,8-bis-benzylthio-octanoic acid in human non-small cell lung cancer xenografts in mice.

FIG. 5 depicts the anti-tumor efficacy of oral 6,8-bis-benzylthio-octanoic acid in human pancreatic cancer xenografts in mice.

FIG. 6 presents X-ray powder diffraction patterns of solid amorphous dispersion formulations of 6,8-bis-benzylthio-octanoic acid with either Eudragit L100 or hydroxypropyl methylcellulose acetate succinate (HPMCAS-M) (top and middle diffraction patterns, respectively), and crystalline 6,8-bis-benzylthio-octanoic acid (bottom diffraction pattern).

### DETAILED DESCRIPTION

The invention provides methods, compositions, and medical kits for treating prostate cancer using 6,8-bis-benzylthio-octanoic acid or a pharmaceutically acceptable salt thereof, including combination therapy with docetaxel. The prostate cancer may be, for example, characterized as an androgen-dependent prostate cancer or an androgen-independent prostate cancer. The 6,8-bis-benzylthio-octanoic acid or a pharmaceutically acceptable salt thereof may be formulated as a pharmaceutical composition, such as a pharmaceutical composition containing an ion pairing agent. When used as part of a combination therapy, the 6,8-bis-benzylthio-octanoic acid or a pharmaceutically acceptable salt thereof may be formulated as a pharmaceutical composition for administration to the patient separate from a pharmaceutical composition containing other agent(s) used in the combination therapy, such as docetaxel. The methods, compositions, and medical kits for treating prostate cancer provide particular benefits to adult, human male patients suffering from prostate cancer. The practice of the present invention employs, unless otherwise indicated, conventional techniques of organic chemistry, pharmacology, and biochemistry. Such techniques are explained in the literature, such as “Comprehensive Organic Synthesis” (B. M. Trost & I. Fleming, eds., 1991-1992); which is incorporated by reference. Various aspects of the invention are set forth below in sections; however, aspects of the invention described in one particular section are not to be limited to any particular section.

### I. Definitions

To facilitate an understanding of the present invention, a number of terms and phrases are defined below.

The terms “a,” “an” and “the” as used herein mean “one or more” and include the plural unless the context is inappropriate

The term “6,8-bis-benzylthio-octanoic acid” refers to CPI-613, i.e., the compound having the chemical structure

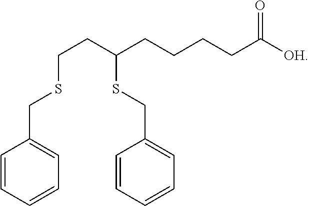 [SMILES: O=C(O)CCCCC(CCSCc1ccccc1)SCc1ccccc1]

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

As used herein, the term “patient” refers to organisms to be treated by the methods of the present invention. Such organisms preferably include, but are not limited to, mammals (e.g., murines, simians, equines (horses), bovines (cattle), porcines, canines, felines, and the like), and most preferably includes humans.

The terms “androgen insensitive,” “androgen independent,” and “androgen resistant” are used interchangeably herein.

The terms “androgen sensitive” and “androgen dependent” are used interchangeably herein.

As used herein, the term “treating” includes any effect, e.g., lessening, reducing, modulating, ameliorating or eliminating, that results in the improvement of the condition, disease, disorder, and the like, or ameliorating a symptom thereof. For example, treatment can include diminishment of a symptom of a disorder or complete eradication of a disorder. As another example, treatment can include slowing the progression of a disease, or preventing or delaying its recurrence, such as maintenance treatment to prevent or delay relapse.

As used herein, the term “pharmaceutical composition” refers to the combination of an active agent with a carrier, inert or active, making the composition especially suitable for diagnostic or therapeutic use in vivo or ex vivo.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” refers to any of the standard pharmaceutical carriers. The compositions also can include stabilizers and preservatives. For examples of carriers, stabilizers, and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Further examples of salts include salts made using the ion pairing agents described in U.S. Pat. No. 8,263,653, the entire disclosure of which is incorporated by reference herein. Still further ion pairing agents can be selected with guidance from Handbook of Pharmaceutical Salts Properties, Selection and Use, UIPAC, Wiley-VCH, P. H. Stahl, ed., the entire disclosure of which is incorporated by reference herein.

Further examples of salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Still other examples of salts include anions of the compounds of the present invention compounded with a suitable cation such as Na⁺, NH₄⁺, and NW₄⁺ (wherein W is a C₁₋₄ alkyl group), and the like. The term “alkyl” is art-recognized, and includes saturated aliphatic groups, including straight-chain alkyl groups and branched-chain alkyl groups.

In certain embodiments, the pharmaceutically acceptable salts are those prepared from the following acids: hydrochloric, hydrobromic, sulfuric, nitric, phosphoric, maleic, acetic, palicylic, p-toluene sulfonic, tartaric, citric, methane sulfonic, formic, malonic, succinic, naphthalene-2-sulfonic, and benzene sulfonic. In certain other embodiments, the pharmaceutically acceptable salts are alkaline metal or alkaline earth salts, such as sodium, potassium or calcium salts of a carboxylic acid group.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Therapeutic Applications

The invention provides methods for treating prostate cancer using 6,8-bis-benzylthio-octanoic acid or a pharmaceutically acceptable salt thereof. The therapeutic methods embrace mono-therapy using 6,8-bis-benzylthio-octanoic acid or a pharmaceutically acceptable salt thereof, as well as combination therapy with a second anti-cancer agent, such as docetaxel. The methods are described in more detail below.

### First Therapeutic Method

One aspect of the invention provides a method for treating prostate cancer. In certain embodiments, the invention provides a method for treating a cancer selected from the group consisting of androgen-dependent prostate cancer and androgen-independent prostate cancer. The method comprises administering to a patient in need thereof a therapeutically effective amount of a first therapeutic agent comprising 6,8-bis-benzylthio-octanoic acid or a pharmaceutically acceptable salt thereof, in order to treat the cancer. The method may be further characterized according to one or more features described herein below.

### Second Therapeutic Method

Another aspect of the invention provides a method for treating prostate cancer comprising administering to a patient in need thereof a therapeutically effective amount of (i) a first therapeutic agent comprising 6,8-bis-benzylthio-octanoic acid or a pharmaceutically acceptable salt thereof and (ii) a second therapeutic agent comprising docetaxel or a pharmaceutically acceptable salt thereof, in order to treat the prostate cancer. The method may be further characterized according to one or more features described herein below.

### Third Therapeutic Method

Another aspect of the invention provides a method for treating prostate cancer comprising administering to a patient in need thereof a unit dosage of a first therapeutic agent comprising 6,8-bis-benzylthio-octanoic acid or a pharmaceutically acceptable salt thereof in an amount ranging from about 150 mg/m² to about 1500 mg/m², to treat the prostate cancer. The method may be further characterized according to one or more features described herein below.

### General Features of the First Therapeutic Method

The above first therapeutic method may be further characterized by additional features, such as the type of cancer treated and whether a second therapeutic agent is administered to the patient.

### Type of Cancer

The therapeutic method can be further characterized according to the type of cancer to be treated. For example, in certain embodiments, the cancer is androgen-dependent prostate cancer. In certain other embodiments, the cancer is androgen-independent prostate cancer.

The cancer may be further characterized according to whether it is metastatic. In certain embodiments, the cancer is metastatic.

### Administering a Second Therapeutic Agent

The therapeutic method may be characterized according to whether a second therapeutic agent is administered to the patient. For example, in certain embodiments, a second therapeutic agent having anti-cancer activity is administered to the patient.

### General Features of the Second Therapeutic Method

The above second therapeutic method may be further characterized by additional features, such as the type of prostate cancer treated and the identity of the second therapeutic agent.

### Type of Prostate Cancer

The therapeutic method can be further characterized according to the type of prostate cancer to be treated. For example, in certain embodiments, the prostate cancer is androgen-dependent prostate cancer. In certain other embodiments, the prostate cancer is androgen-independent prostate cancer.

The prostate cancer may be further characterized according to whether it is metastatic. In certain embodiments, the prostate cancer is metastatic.

### Identity of the Second Therapeutic Agent

The therapeutic method may be further characterized according to the identity of the second therapeutic agent. For example, in certain embodiments, the second therapeutic agent is docetaxel.

### Dose, Timing, and Route for Administration of the Second Therapeutic Agent

The therapeutic method may be characterized according to features of administration of the second therapeutic agent. For instance, in certain embodiments, the therapeutic method may be characterized according to the dose of the second therapeutic agent administered to the patient. Accordingly, in certain embodiments, the second therapeutic agent is administered at a dosage ranging from about 50 mg/m² to about 100 mg/m². In certain embodiments, the second therapeutic agent is administered at a dosage of about 75 mg/m².

The therapeutic method may also be characterized according to the frequency of administration of the second therapeutic agent. For example, in certain embodiments, the second therapeutic agent is administered once per any three week period.

The therapeutic method may be further characterized according to the route of administration of the second therapeutic agent. For example, in certain embodiments, the second therapeutic agent is administered by intravenous administration.

The therapeutic method may also be characterized according to the scheduled dosing cycle for administration of the docetaxel. For example, in certain embodiments, the docetaxel is administered on the first day of a three week cycle. In certain embodiments, the docetaxel is administered at a dose from about 50 mg/m² to about 100 mg/m² on the first day of a three week cycle. In certain embodiments, the docetaxel is administered at a dosage of about 75 mg/m² on the first day of a three week cycle. In certain embodiments the docetaxel is administered as a one hour infusion on the first day of a three week cycle. In certain embodiments, the scheduled cycle is repeated at least once. In certain embodiments, the method of the present invention comprises treatment with up to 10 scheduled cycles.

### General Features of the First and Second Therapeutic Methods

The above first and second therapeutic methods may be further characterized by additional features, such as the unit dosage, timing, and route for administration of the first therapeutic agent.

### Unit Dosage, Timing, and Route for Administration of the First Therapeutic Agent

The therapeutic methods may be characterized according to features of administration of the first therapeutic agent. For instance, in certain embodiments, the therapeutic methods may be characterized according to the unit dosage of the first therapeutic agent administered to the patient. Accordingly, in certain embodiments, the first therapeutic agent is administered at a unit dosage ranging from about 150 mg/m² to about 1500 mg/m². In certain other embodiments, the first therapeutic agent is administered at a unit dosage ranging from about 150 mg/m² to about 1300 mg/m². In certain other embodiments, the first therapeutic agent is administered at a unit dosage ranging from about 500 mg/m² to about 1000 mg/m². In certain other embodiments, the first therapeutic agent is administered at a unit dosage of about 500 mg/m².

The therapeutic methods may be further characterized according to the route of administration of the first therapeutic agent. For example, in certain embodiments, the first therapeutic agent is administered orally. In certain other embodiments, the first therapeutic agent is administered by intravenous administration.

The therapeutic methods may also be characterized according to the frequency of administration of the first therapeutic agent. For example, in certain embodiments, the first therapeutic agent is administered to the patient no more frequently than once per day. In certain embodiments, the first therapeutic agent is administered to the patient twice per week. In certain other embodiments, the first therapeutic agent is administered to the patient once per week. In certain other embodiments, the first therapeutic agent is administered to the patient once every two weeks. In certain other embodiments, the first therapeutic agent is administered to the patient no more frequently than once every two weeks.

The therapeutic method may also be characterized according to the scheduled dosing cycle for administration of the first therapeutic agent. For example, in certain embodiments, the first therapeutic agent is administered in a four week cycle in which the first therapeutic agent is administered twice per week during the first three weeks followed by one week off. In certain embodiments, the first therapeutic agent is administered on days 1, 8, and 15 of a four week cycle. In certain embodiments, the first therapeutic agent is administered on days 1 and 3 of a two week cycle. In certain embodiments, the first therapeutic agent is administered at a dose of about 150 mg/m² to about 1500 mg/m² twice per week during the first three weeks followed by one week off. In certain embodiments, the first therapeutic agent is administered at a dose of about 150 mg/m² to about 1500 mg/m² on days 1, 8, and 15 of a four week cycle. In certain embodiments, the first therapeutic agent is administered at a dose of about 150 mg/m² to about 1500 mg/m² on days 1 and 3 of a two week cycle. In certain embodiments, the first therapeutic agent is administered at a dose of about 500 mg/m² to about 100 mg/m² twice per week during the first three weeks followed by one week off. In certain embodiments, the first therapeutic agent is administered at a dose of about 500 mg/m² to about 100 mg/m² on days 1, 8, and 15 of a four week cycle. In certain embodiments, the first therapeutic agent is administered at a dose of about 500 mg/m² to about 1000 mg/m² on days 1 and 3 of a two week cycle. In certain embodiments, the first therapeutic agent is administered at a dose of about 150 mg/m² to about 500 mg/m² twice per week during the first three weeks followed by one week off. In certain embodiments, the first therapeutic agent is administered at a dose of about 150 mg/m² to about 500 mg/m² on days 1, 8, and 15 of a four week cycle. In certain embodiments, the first therapeutic agent is administered at a dose of about 150 mg/m² to about 500 mg/m² on days 1 and 3 of a two week cycle. In certain embodiments, the first therapeutic agent is administered at a dose of about 500 mg/m² twice per week during the first three weeks followed by one week off. In certain embodiments, the first therapeutic agent is administered at a dose of about 500 mg/m² on days 1, 8, and 15 of a four week cycle. In certain embodiments, the first therapeutic agent is administered at a dose of about 500 mg/m² on days 1 and 3 of a two week cycle. In certain embodiments, each of the above doses of the first therapeutic agent is administered as a two hour IV infusion. In certain embodiments, the scheduled cycle is repeated at least once. In certain embodiments, the method of the present invention comprises treatment with up to 10 scheduled cycles.

The first therapeutic agent may be orally administered to the patient. The dose and schedule will vary based on, e.g., the characteristics of the patient's cancer and whether another therapeutic agent will be administered in combination, and can be readily determined by those of ordinary skill in the art in view of the guidance provided herein. In certain embodiments, the dose and schedule is adapted based on the doses and schedules used intravenously with 6,8-bis-benzylthio-octanoic acid or a pharmaceutically acceptable salt thereof, such as those set forth herein. In certain embodiments, the dose is the maximum tolerated dose.

An advantage of oral dosing of the first therapeutic agent is that it permits substantially increased dosing flexibility as compared to IV. In the prior art, 6,8-bis-benzylthio-octanoic acid is formulated as a 50 mg/mL solution in 1 M (150 mg/mL) aqueous triethanolamine, which is diluted from 50 mg/mL to as low as 4 mg/mL (e.g., 12.5 mg/mL) with sterile 5% dextrose for injection (D5W) prior to administration as an IV infusion over 30-120 minutes via a central venous catheter. Such an infusion is inconvenient for patients and effectively precludes regimens involving frequent and/or prolonged dosing. Since the half-life of the first therapeutic agent after IV dosing is only about 1-2 hours (Pardee, T. S. et al., Clin Cancer Res. 2014, 20, 5255-64), more frequent and/or prolonged dosing could advantageously be used to increase the patient's exposure to the first therapeutic agent.

For example, a possible IV schedule for the treatment of prostate cancer involves administering the first therapeutic agent twice per week for the first three weeks of a four week cycle. If administered orally, the practitioner would have more flexibility with respect to the first therapeutic agent dose and schedule. The first therapeutic agent could be orally administered in a single daily dose twice per week for the first three weeks of a four week cycle as in the IV schedule. Alternatively, the first therapeutic agent could be administered in two or more (e.g., three, four, or five) divided doses on two days per week for the first three weeks of a four week cycle and/or the first therapeutic agent could be administered on fewer or additional days of the cycle, up to and including every day.

Another advantage of oral dosing is that it makes maintenance therapy feasible. For example, a patient who is treated successfully with first line therapy—with or without the first therapeutic agent—and whose cancer is in partial or complete remission, may be treated orally with the first therapeutic agent on a chronic basis in order to delay or prevent recurrence. The maintenance treatment may involve, for example, one, two, three, four, or five doses per day of the first therapeutic agent on a regular basis, such as daily or weekly.

In certain embodiments, the first therapeutic agent is orally administered at a dose of about 1 mg to about 10,000 mg on each day it is administered. The daily dose may be administered in one dose or divided into two or more doses, such as three, four, or five doses. In certain embodiments, the daily dose is about 10 mg to about 7,500 mg. In certain embodiments, the daily dose is about 100 mg to about 5,000 mg. In certain embodiments, the daily dose is about 200 mg to about 4,000 mg. In certain embodiments, the daily dose is about 300 mg to about 3,000 mg. In certain embodiments, the daily dose is about 400 mg to about 2,500 mg. In certain embodiments, the daily dose is about 500 mg to about 2,000 mg. In certain embodiments, the daily dose is about 100 mg, 200 mg, 300 mg, 400 mg, 500 mg, 600 mg, 700 mg, 800 mg, 900 mg, 1,000 mg, 1,250 mg, 1,500 mg, 1,750 mg, 2,000 mg, 2,500 mg, 3,000 mg, 3,500 mg, 4,000 mg, 4,500 mg, 5,000 mg, 6,000 mg, 7,000 mg, 8,000 mg, 9,000 mg, or 10,000 mg.

In certain embodiments, a dosing cycle is repeated at least once. In certain embodiments, the method of the present invention comprises treatment with two cycles or more. In certain embodiments, the method of the present invention comprises treatment with three cycles or more. In certain embodiments, the method of the present invention comprises treatment with four cycles or more. In certain embodiments, the method of the present invention comprises treatment with five cycles or more. In certain embodiments, the method of the present invention comprises treatment with six cycles or more. In certain embodiments, the method of the present invention comprises treatment with seven cycles or more. In certain embodiments, the method of the present invention comprises treatment with eight cycles or more. In certain embodiments, the method of the present invention comprises treatment with nine cycles or more. In certain embodiments, the method of the present invention comprises treatment with ten cycles or more. In certain embodiments, the method of the present invention comprises regular treatment with the first therapeutic agent, including on a daily or weekly basis, for an extended period of time, such as one year, two years, three years, or longer.

### General Features of the Third Therapeutic Method

The above third therapeutic method may be further characterized by additional features, such as the amount, timing, and route for administration of the unit dosage.

### Amount, Timing, and Route for Administration of the Unit Dosage

The therapeutic method may be characterized according to features of administration of the unit dosage. For instance, in certain embodiments, the therapeutic methods may be characterized according to the amount of the unit dosage administered to the patient. Accordingly, in certain embodiments, the unit dosage is from about 500 mg/m² to about 1000 mg/m² of 6,8-bis-benzylthio-octanoic acid or a pharmaceutically acceptable salt thereof. In certain other embodiments, the unit dosage is about 500 mg/m² of 6,8-bis-benzylthio-octanoic acid or a pharmaceutically acceptable salt thereof.

The therapeutic method may also be characterized according to the frequency of administration of the unit dosage. For example, in certain embodiments, the unit dosage is administered to the patient no more frequently than once per day. In certain embodiments, the unit dosage is administered to the patient twice per week. In certain other embodiments, the unit dosage is administered to the patient once per week. In certain other embodiments, the unit dosage is administered to the patient once every two weeks. In certain other embodiments, the unit dosage is administered to the patient no more frequently than once every two weeks.

The therapeutic method may be further characterized according to the route of administration of the unit dosage. For example, in certain embodiments, the unit dosage is administered intravenously to the patient. In certain embodiments, the unit dosage is administered orally to the patient.

The therapeutic method may also be characterized according to the scheduled dosing cycle for administration of the 6,8-bis-benzylthio-octanoic acid or pharmaceutically acceptable salt thereof. For example, in certain embodiments, the 6,8-bis-benzylthio-octanoic acid or pharmaceutically acceptable salt thereof is administered in a four week cycle in which the 6,8-bis-benzylthio-octanoic acid or pharmaceutically acceptable salt thereof is administered twice per week during the first three weeks followed by one week off. In certain embodiments, the 6,8-bis-benzylthio-octanoic acid or pharmaceutically acceptable salt thereof is administered on days 1, 8, and 15 of a four week cycle. In certain embodiments, the 6,8-bis-benzylthio-octanoic acid or pharmaceutically acceptable salt thereof is administered on days 1 and 3 of a two week cycle. In certain embodiments, the 6,8-bis-benzylthio-octanoic acid or pharmaceutically acceptable salt thereof is administered at a dose of about 150 mg/m² to about 1500 mg/m² twice per week during the first three weeks followed by one week off. In certain embodiments, the 6,8-bis-benzylthio-octanoic acid or pharmaceutically acceptable salt thereof is administered at a dose of about 150 mg/m² to about 1500 mg/m² on days 1, 8, and 15 of a four week cycle. In certain embodiments, the 6,8-bis-benzylthio-octanoic acid or pharmaceutically acceptable salt thereof is administered at a dose of about 150 mg/m² to about 1500 mg/m² on days 1 and 3 of a two week cycle. In certain embodiments, the 6,8-bis-benzylthio-octanoic acid or pharmaceutically acceptable salt thereof is administered at a dose of about 500 mg/m² to about 100 mg/m² twice per week during the first three weeks followed by one week off. In certain embodiments, the 6,8-bis-benzylthio-octanoic acid or pharmaceutically acceptable salt thereof is administered at a dose of about 500 mg/m² to about 100 mg/m² on days 1, 8, and 15 of a four week cycle. In certain embodiments, the 6,8-bis-benzylthio-octanoic acid or pharmaceutically acceptable salt thereof is administered at a dose of about 500 mg/m² to about 1000 mg/m² on days 1 and 3 of a two week cycle. In certain embodiments, the 6,8-bis-benzylthio-octanoic acid or pharmaceutically acceptable salt thereof is administered at a dose of about 150 mg/m² to about 500 mg/m² twice per week during the first three weeks followed by one week off. In certain embodiments, the 6,8-bis-benzylthio-octanoic acid or pharmaceutically acceptable salt thereof is administered at a dose of about 150 mg/m² to about 500 mg/m² on days 1, 8, and 15 of a four week cycle. In certain embodiments, the 6,8-bis-benzylthio-octanoic acid or pharmaceutically acceptable salt thereof is administered at a dose of about 150 mg/m² to about 500 mg/m² on days 1 and 3 of a two week cycle. In certain embodiments, the 6,8-bis-benzylthio-octanoic acid or pharmaceutically acceptable salt thereof is administered at a dose of about 500 mg/m² twice per week during the first three weeks followed by one week off. In certain embodiments, the 6,8-bis-benzylthio-octanoic acid or pharmaceutically acceptable salt thereof is administered at a dose of about 500 mg/m² on days 1, 8, and 15 of a four week cycle. In certain embodiments, the 6,8-bis-benzylthio-octanoic acid or pharmaceutically acceptable salt thereof is administered at a dose of about 500 mg/m² on days 1 and 3 of a two week cycle. In certain embodiments, each of the above doses of the 6,8-bis-benzylthio-octanoic acid or pharmaceutically acceptable salt thereof is administered as a two hour IV infusion. In certain embodiments, the scheduled cycle is repeated at least once. In certain embodiments, the method of the present invention comprises treatment with up to 10 scheduled cycles.

### Additional General Features of the First, Second, and Third Therapeutic Method

The above first, second, and third therapeutic methods may be further characterized by additional features, such as the patients to be treated and the form of the first therapeutic agent.

### Patients for Treatment

The therapeutic methods may be further characterized according to the patient to be treated. Preferably, the patient is a human being. In certain embodiments, the patient is an adult human. In certain other embodiments, the patient is at least partially refractory to docetaxel.

### Form of the First Therapeutic Agent

The methods may be further characterized according to the form of the first therapeutic agent. For example, in certain embodiments, the first therapeutic agent is 6,8-bis-benzylthio-octanoic acid or a pharmaceutically acceptable salt thereof. In certain other embodiments, the first therapeutic agent is 6,8-bis-benzylthio-octanoic acid.

The first therapeutic agent may be formulated in a pharmaceutical composition. In certain embodiments, the first therapeutic agent is administered in the form of a pharmaceutical composition comprising 6,8-bis-benzylthio-octanoic acid and a pharmaceutically acceptable carrier. In certain other embodiments, the first therapeutic agent is administered in the form of a pharmaceutical composition comprising 6,8-bis-benzylthio-octanoic acid and an ion pairing agent. In certain embodiments, the first therapeutic agent is administered in the form of a pharmaceutical composition comprising 6,8-bis-benzylthio-octanoic acid and triethanolamine. In certain other embodiments, the pharmaceutical composition further comprises dextrose and water.

Exemplary ion pairing agents that may be used include, for example, a tertiary amine (such as triethanolamine), other amines such as diethanolamine, monoethanolamine, mefenamic acid and tromethamine, and combinations thereof. In certain embodiments, the ion pairing agent is an organic Bronsted base. In certain other embodiments, the ion pairing agent is an amine compound. In yet other embodiments, the ion pairing agent is a monoalkylamine, dialkylamine, trialkylamine, amino-substituted aliphatic alcohol, hydroxymonoalkylamine, hydroxydialkylamine, hydroxytrialkylamine, amino-substituted heteroaliphatic alcohol, alkyldiamine, substituted alkyldiamine, or optionally substituted heteroaryl group containing at least one ring nitrogen atom.

Additional exemplary ion pairing agents include, for example, polyethyleneimine, polyglutamic acid, ammonia, L-arginine, benethamine benzathine, betaine, calcium hydroxide, choline, deanol, diethanolamine(2,2′-iminobis(ethanol)), diethylamine, 2-(diethylamino)-ethanol, ethanolamine, ethylenediamine, N-methyl-glucamine, hydrabamine, 1H-imidazole, lysine, magnesium hydroxide, 4-(2-hydroxyethyl)-morpholine, piperazine, potassium hydroxide, 1-(2-hydroxyethyl)-pyrrolidine, sodium hydroxide, triethanolamine (2,2′,2″-nitrilotris(ethanol)), tromethamine, and zinc hydroxide. In certain other embodiments, the ion pairing agent is diisopropanolamine, 3-amino-1-propanol, meglumine, morpholine, pyridine, niacinamide, tris(hydroxymethyl)aminomethane, 2-((2-dimethylamino)ethoxy)ethanol, 2-(dimethylamino)ethanol, 1-(2-hydroxyethyl)pyrrolidine, or ammonium hydroxide. In certain other embodiments, the ion pairing agent is an alkali metal hydroxide or alkaline earth metal hydroxide, such as, for example, cesium hydroxide.

In certain embodiments, the first therapeutic agent has a purity of at least about 50% (w/w). In certain embodiments, the first therapeutic agent has a purity of at least about 60% (w/w). In certain embodiments, the first therapeutic agent has a purity of at least about 70% (w/w). In certain embodiments, the first therapeutic agent has a purity of at least about 80% (w/w). In certain embodiments, the first therapeutic agent has a purity of at least about 90% (w/w). In certain embodiments, the first therapeutic agent has a purity of at least about 95% (w/w). In certain embodiments, the first therapeutic agent has a purity of at least about 96% (w/w). In certain embodiments, the first therapeutic agent has a purity of at least about 97% (w/w). In certain embodiments, the first therapeutic agent has a purity of at least about 98% (w/w). In certain embodiments, the first therapeutic agent has a purity of at least about 99% (w/w).

In certain other embodiments, the first therapeutic agent is administered in the form of a pharmaceutical composition comprising 6,8-bis-benzylthio-octanoic acid and triethanolamine. Such pharmaceutical compositions may be further characterized according to the mole ratio of the triethanolamine to 6,8-bis-benzylthio-octanoic acid. In certain embodiments, the mole ratio of triethanolamine to 6,8-bis-benzylthio-octanoic acid is in the range of about 10:1 to about 1:10, about 10:1 to about 5:1, or about 8:1. In certain embodiments, the mole ratio of triethanolamine to 6,8-bis-benzylthio-octanoic acid is about 8:1.

### Exemplary Dosing Amounts & Treatment Cycles for the First and Second Therapeutic Methods

Generally, a therapeutic agent, e.g., 6,8-bis-benzylthio-octanoic acid or a pharmaceutically acceptable salt thereof, is delivered to the patient in a therapeutically effective amount. The therapeutically effective amount of a therapeutic agent may vary with the activity of the specific agent employed; the metabolic stability and length of action of that agent; the species, age, body weight, general health, dietary status, sex and diet of the subject; the mode and time of administration; rate of excretion; drug combination, if any; and extent of presentation and/or severity of the particular condition being treated. The precise dosage can be determined, may involve one or several administrations per day, in whichever order is necessary or desirable, to yield the desired results, and the dosage may be adjusted by the individual practitioner to achieve a desired effect. The treatment may involve one or several administrations on one or more days, and the dosage may be adjusted by the individual practitioner to achieve a desired effect. Preferably, the dosage amount of the agent(s) used should be sufficient to interact solely with disease cells (e.g., tumor cells), leaving normal cells comparatively unharmed (e.g., essentially unharmed).

The dosage amount may be administered in a single dose or in the form of individual divided doses, such as from one to four or more times per day. In certain embodiments, the daily dosage amount is administered in a single dose. In the event that the response in a subject is insufficient at a certain dose, even higher doses (or effective higher doses by a different, more localized delivery route) may be employed to the extent of patient tolerance.

Components in a combination therapy may be administered in a particular order and/or according to a treatment cycle, such as on the same or different days. For example, in certain embodiments, at least one dose of the first therapeutic agent is administered to the patient prior to administering the second therapeutic agent, such as on an earlier day in a treatment cycle. In certain other embodiments, active components of the combination therapy may be administered on the same day of a treatment cycle, for example being co-administered simultaneously. In certain embodiments, at least one dose of a second therapeutic agent is administered to the patient prior to administering the 6,8-bis-benzylthio-octanoic acid or a pharmaceutically acceptable salt thereof, such as on an earlier day in a treatment cycle. In certain embodiments, active components of the combination therapy may be co-administered in a predetermined manner, ratio, and order of addition so as to comprise a treatment cycle. In certain embodiments, treatment cycles may be repeated in order to maximize benefit to the patient.

### Treatment Efficacy and Safety

The therapeutic method of the present invention may be further characterized by the efficacy and safety of the treatment. Preferably, the method provides an acceptable safety profile, with the benefit of treatment outweighing the risk. When tested in a phase II or phase III clinical trial of at least 10 patients with prostate cancer, the method of the present invention preferably provides an overall response rate of at least about 10%, a duration of response of at least about 1 month, progression-free survival (PFS) of at least about 1 month, and/or overall survival (OS) of at least about 1 month. Preferably, the phase II or phase III clinical trial comprises at least 15 patients. More preferably, the phase II or phase III clinical trial comprises at least 20 patients. More preferably, the phase II or phase III clinical trial comprises at least 25 patients. More preferably, the phase II or phase III clinical trial comprises at least 50 patients. More preferably, the phase II or phase III clinical trial comprises at least 100 patients. More preferably, the phase II or phase III clinical trial comprises at least 200 patients. More preferably, the phase II or phase III clinical trial comprises at least 300 patients. More preferably, the phase II or phase III clinical trial comprises at least 400 patients. More preferably, the phase II or phase III clinical trial comprises at least 500 patients. Preferably, the method of the present invention provides an overall response rate of at least about 20% in patients. More preferably, the method of the present invention provides an overall response rate of at least about 30%. More preferably, the method of the present invention provides an overall response rate of at least about 40%. More preferably, the method of the present invention provides an overall response rate of at least about 50%. More preferably, the method of the present invention provides an overall response rate of at least about 60%. More preferably, the method of the present invention provides an overall response rate of at least about 70%. More preferably, the method of the present invention provides an overall response rate of at least about 80%. More preferably, the method of the present invention provides an overall response rate of at least about 90%. Preferably, the method of the present invention provides a duration of response, PFS, and/or OS of at least about 2 months. Preferably, the method of the present invention provides a duration of response, PFS, and/or OS of at least about 3 months. Preferably, the method of the present invention provides a duration of response, PFS, and/or OS of at least about 4 months. Preferably, the method of the present invention provides a duration of response, PFS, and/or OS of at least about 5 months. Preferably, the method of the present invention provides a duration of response, PFS, and/or OS of at least about 6 months. Preferably, the method of the present invention provides a duration of response, PFS, and/or OS of at least about 7 months. Preferably, the method of the present invention provides a duration of response, PFS, and/or OS of at least about 8 months. Preferably, the method of the present invention provides a duration of response, PFS, and/or OS of at least about 9 months. Preferably, the method of the present invention provides a duration of response, PFS, and/or OS of at least about 10 months. Preferably, the method of the present invention provides a duration of response, PFS, and/or OS of at least about 11 months. Preferably, the method of the present invention provides a duration of response, PFS, and/or OS of at least about 12 months. Preferably, the method of the present invention provides a duration of response, PFS, and/or OS of at least about 14 months. Preferably, the method of the present invention provides a duration of response, PFS, and/or OS of at least about 16 months. Preferably, the method of the present invention provides a duration of response, PFS, and/or OS of at least about 18 months. Preferably, the method of the present invention provides a duration of response, PFS, and/or OS of at least about 20 months. Preferably, the method of the present invention provides a duration of response, PFS, and/or OS of at least about 24 months. In certain embodiments, the overall response rate, duration of response, and progression-free survival mentioned above are measured in a phase II clinical trial. In certain embodiments, the overall response rate, duration of response, and progression-free survival mentioned above are measured in a phase III clinical trial.

The methods desirably administer a therapeutically effective amount of the indicated compound(s) to the patient. A therapeutically effective amount can be determined based on guidance herein and may, for instance, be an amount of a compound sufficient to inhibit, halt, or cause an improvement in a disorder or condition being treated in a particular subject or subject population. For example, a therapeutically effective amount can be an amount of drug sufficient to slow the progression of a disease, or to prevent or delay its recurrence, such as maintenance treatment to prevent or delay relapse. In a human or other mammal, a therapeutically effective amount can be determined experimentally in a laboratory or clinical setting, or may be the amount required by the guidelines of the United States Food and Drug Administration, or equivalent foreign agency, for the particular disease and subject being treated. It should be appreciated that determination of proper dosage forms, dosage amounts, and routes of administration is within the level of ordinary skill in the pharmaceutical and medical arts.

### IV. Medical Kits

Another aspect of the invention provides medical kits containing a therapeutic agent and/or pharmaceutical composition described herein, along with instructions for using the kits to treat a disorder described herein. In certain embodiments, the medical kit comprises (i) a first therapeutic agent comprising 6,8-bis-benzylthio-octanoic acid or a pharmaceutically acceptable salt thereof, and (ii) instructions for treating a cancer selected from the group consisting of androgen-dependent prostate cancer and androgen-independent prostate cancer using said first therapeutic agent. The medical kit may be further characterized according to one or more of the features described herein in connection with the First Therapeutic Method.

Another aspect of the invention provides a medical kit for treating prostate cancer, wherein the kit comprises (i) a first therapeutic agent comprising 6,8-bis-benzylthio-octanoic acid or a pharmaceutically acceptable salt thereof, and (ii) instructions for treating prostate cancer using the first therapeutic agent in combination with a second therapeutic agent comprising docetaxel or a pharmaceutically acceptable salt thereof. The medical kit may be further characterized according to one or more of the features described herein in connection with the Second Therapeutic Method.

Another aspect of the invention provides a medical kit for treating prostate cancer, wherein the kit comprises (i) a first therapeutic agent comprising 6,8-bis-benzylthio-octanoic acid or a pharmaceutically acceptable salt thereof, and (ii) instructions for treating prostate cancer using a dosage of the first therapeutic agent ranging from about 500 mg/m² to about 1000 mg/m². The medical kit may be further characterized according to one or more of the features described herein in connection with the Third Therapeutic Method.

### V. Pharmaceutical Compositions

Therapeutic agents described herein may be formulated as a pharmaceutical composition comprising one or more therapeutic agents and a pharmaceutically acceptable carrier. For example, the first therapeutic agent can be formulated as a pharmaceutical composition that, for example, optionally further contains a further anti-cancer agent. A pharmaceutical composition that contains both a first therapeutic agent and a second therapeutic agent may be referred to as a co-formulated composition.

In certain embodiments of the present invention, a therapeutic agent may be formulated as a pharmaceutically-acceptable oil; liposome; oil-water or lipid-oil-water emulsion or nanoemulsion; liquid; or salt, crystalline form, or other solid form delivered in a tablet or capsule. To facilitate such formulations, the therapeutic agent may be combined with a pharmaceutically-acceptable carrier or excipient therefor. Examples of pharmaceutically-acceptable carriers are well known in the art and include those conventionally used in pharmaceutical compositions, such as salts, lipids, buffers, chelating agents, flavorants, colorants, preservatives, absorption promoters to enhance bioavailability, antimicrobial agents, and combinations thereof, optionally in combination with other therapeutic ingredients.

As described in detail below, the pharmaceutical compositions may be specially formulated for administration in solid or liquid form, including those adapted for parenteral administration, for example, by subcutaneous, intramuscular, intravenous or epidural injection as, for example, a sterile solution or suspension, or sustained-release formulation.

Further examples of pharmaceutical formulations of the CPI-613 or pharmaceutically acceptable salt thereof are described in U.S. Pat. No. 8,263,653, the entire disclosure of which is incorporated by reference herein.

Methods of preparing pharmaceutical formulations or pharmaceutical compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

In certain embodiments, one or more of the therapeutic agents are administered by intraparenteral administration. In certain other embodiments, one or more of the therapeutic agents are formulated for inhalational, oral, topical, transdermal, nasal, ocular, pulmonary, rectal, transmucosal, intravenous, intramuscular, subcutaneous, intraperitoneal, intrathoracic, intrapleural, intrauterine, intratumoral, or infusion methodologies or administration, or combinations of any thereof, in the form of aerosols, sprays, powders, gels, lotions, creams, suppositories, ointments, and the like. As indicated above, if such a formulation is desired, other additives known in the art may be included to impart the desired consistency and other properties to the formulation.

In certain embodiments, the pharmaceutical composition comprising the first therapeutic agent is an oral dosage form, such as a dry oral dosage form. In certain embodiments, the pharmaceutical composition comprising the first therapeutic agent is an oral dosage form chosen from tablet, pill, capsule, caplet, powder, granule, solution, suspension, and gel. Oral dosage forms may include pharmaceutically acceptable excipients, such as carriers, diluents, stabilizers, plasticizers, binders, glidants, disintegrants, bulking agents, lubricants, plasticizers, colorants, film formers, flavoring agents, preservatives, dosing vehicles, and any combination of any of the foregoing. Pharmaceutically acceptable excipients are determined in part by the particular composition being administered, as well as by the particular dosing schedule. Accordingly, there is a wide variety of suitable formulations of pharmaceutical compositions of the present invention (see, e.g., Remington: The Science and Practice of Pharmacy, 20th ed., Gennaro et al. Eds., Lippincott Williams and Wilkins, 2000).

The oral pharmaceutical composition comprising the first therapeutic agent will generally include at least one inert excipient. Excipients include pharmaceutically compatible binding agents, lubricants, wetting agents, disintegrants, and the like. Tablets, pills, capsules, troches and the like can contain any of the following excipients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain a liquid excipient such as a fatty oil. In addition, dosage unit forms can contain various other materials that modify the physical form of the dosage unit, for example, coatings of sugar, shellac, or enteric agents. Further, a syrup may contain, in addition to the active compounds, sucrose as a sweetening agent and certain preservatives, dyes, colorings, and flavorings. In certain embodiments, the oral pharmaceutical composition comprising the first therapeutic agent comprises an excipient in an amount of about 5% to about 99%, such as about 10% to about 85%, by weight of the composition, with the first therapeutic agent comprising the remainder. In certain embodiments, pharmaceutically acceptable excipients comprise about 20% to about 80% of the total weight of the composition. In certain embodiments, the pharmaceutical composition comprises the first therapeutic agent in an amount of at least about 40% by weight of the composition, with one or more excipients comprising the remainder. In certain embodiments, the pharmaceutical composition comprises the first therapeutic agent in an amount of at least about 50% by weight of the composition. In certain embodiments, the pharmaceutical composition comprises the first therapeutic agent in an amount of at least about 60% by weight of the composition. In certain embodiments, the pharmaceutical composition comprises the first therapeutic agent in an amount of at least about 70% by weight of the composition. In certain embodiments, the pharmaceutical composition comprises the first therapeutic agent in an amount of at least about 80% by weight of the composition. In certain embodiments, the pharmaceutical composition comprises the first therapeutic agent in an amount of at least about 90% by weight of the composition.

Diluents for solid oral pharmaceutical compositions comprising the first therapeutic agent include, but are not limited to, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit), potassium chloride, powdered cellulose, sodium chloride, sorbitol and talc.

Binders for solid oral pharmaceutical compositions comprising the first therapeutic agent include, but are not limited to, acacia, tragacanth, sucrose, glucose, alginic acid, carbomer (e.g. Carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch. In certain embodiments, the pharmaceutical composition comprises a binder in an amount of about 0.5% to about 25%, such as about 0.75% to about 15%, by weight of the composition. In certain embodiments, the pharmaceutical composition comprises a binder in an amount of about 1% to about 10% by weight of the composition.

The dissolution rate of a compacted solid pharmaceutical composition in a patient's stomach may be increased by the addition of a disintegrant to the composition comprising the first therapeutic agent. Disintegrants include, but are not limited to, alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, powdered cellulose, pregelatinized starch, sodium alginate, sodium starch glycolate (e.g. EXPLOTAB®) and starch. In certain embodiments, the pharmaceutical composition comprises a disintegrant in an amount of about 0.2% to about 30%, such as about 0.2% to about 10%, by weight of the composition. In certain embodiments, the pharmaceutical composition comprises a disintegrant in an amount of about 0.2% to about 5% by weight of the composition.

The oral pharmaceutical composition comprising the first therapeutic agent optionally comprises one or more pharmaceutically acceptable wetting agents. Such wetting agents are preferably selected to maintain the API in close association with water, a condition that is believed to improve bioavailability of the composition. Non-limiting examples of surfactants that can be used as wetting agents include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene, caprylic/capric mono- and diglycerides (e.g., Labrasol™ of Gattefosse), polyoxyethylene castor oil and polyoxyethylene hydrogenated castor oil; polyoxyethylene alkyl ethers, for example polyoxyethylene cetostearyl ether, polyoxyethylene fatty acid esters, for example polyoxyethylene stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. In certain embodiments, the pharmaceutical composition comprising the first therapeutic agent comprises a wetting agent in an amount of about 0.25% to about 15%, such as about 0.4% to about 10%, by weight of the composition. In certain embodiments, the pharmaceutical composition comprises a wetting agent in an amount of about 0.5% to about 5% by weight of the composition. In certain embodiments, the pharmaceutical composition comprises a wetting agent that is an anionic surfactant. In certain embodiments, the pharmaceutical composition comprises sodium lauryl sulfate as a wetting agent. In certain embodiments, the pharmaceutical composition comprises sodium lauryl sulfate in an amount of about 0.25% to about 7%, such as about 0.4% to about 4%, by weight of the composition. In certain embodiments, the pharmaceutical composition comprises sodium lauryl sulfate in an amount of about 0.5% to about 2% by weight of the composition.

Lubricants (e.g., anti-adherents or glidants) can be added to improve the flow properties of solid oral compositions comprising the first therapeutic agent and/or to reduce friction between the composition and equipment during compression of tablet formulations. Excipients that may function as lubricants include, but are not limited to, colloidal silicon dioxide, magnesium trisilicate, powdered cellulose, starch, talc and tribasic calcium phosphate. Suitable lubricants further include glyceryl behapate (e.g., Compritol™ 888 of Gattefosse); stearic acid and salts thereof, including magnesium, calcium and sodium stearates; zinc stearate; glyceryl monostearate; glyceryl palmitostearate; hydrogenated castor oil; hydrogenated vegetable oils (e.g., Sterotex™ of Abitec); waxes; boric acid; sodium benzoate; sodium acetate; sodium stearyl fumarate; sodium fumarate; sodium chloride; DL-leucine; PEG (e.g., Carbowax™ 4000 and Carbowax™ 6000 of the Dow Chemical Company); sodium oleate; sodium lauryl sulfate; and magnesium lauryl sulfate. In certain embodiments, an oral pharmaceutical composition comprising the first therapeutic agent comprises a lubricant in an amount of about 0.1% to about 10%, such as about 0.2% to about 8%, by weight of the composition. In certain embodiments, the pharmaceutical composition comprises a lubricant in an amount of about 0.25% to about 5% by weight of the composition. In certain embodiments, the pharmaceutical composition comprises magnesium stearate as a lubricant. In certain embodiments, the pharmaceutical composition comprises colloidal silicon dioxide. In certain embodiments, the pharmaceutical composition comprises talc. In certain embodiments, the composition comprises magnesium stearate or talc in an amount of about 0.5% to about 2% by weight of the composition.

Flavoring agents and flavor enhancers make the oral dosage form comprising the first therapeutic agent more palatable to the patient. Common flavoring agents and flavor enhancers for pharmaceutical products that may be included in the composition of the present invention include maltol, vanillin, ethyl vanillin, menthol, citric acid, fumaric acid ethyl maltol, and tartaric acid.

Compositions may also be colored using any pharmaceutically acceptable colorant to improve their appearance and/or facilitate patient identification of the product and unit dosage level.

Selection of excipients and the amounts to use may be readily determined by formulation scientists based upon experience and consideration of standard procedures and reference works in the field. The solid oral compositions comprising the first therapeutic agent of the present invention include powders, granulates, aggregates and compacted compositions. The dosages may be conveniently presented in unit dosage form and prepared by any of the methods well-known in the pharmaceutical arts. Dosage forms include solid dosage forms like tablets, pills, powders, caplets, granules, capsules, sachets, troches and lozenges. In certain embodiments, the pharmaceutical composition comprising the first therapeutic agent is a tablet. In certain embodiments, the pharmaceutical composition comprising the first therapeutic agent is a spray-dried dispersion. In certain embodiments, the pharmaceutical composition comprising the first therapeutic agent is a spray-dried dispersion comprising at least one polymer chosen from polyacrylate, polymethacrylate, poly(vinylpyrrolidone), hydroxypropyl methyl cellulose (HPMC), cellulose acetate phthalate (CAP), and hydroxypropyl methylcellulose acetate succinate (HPMCAS-M). In certain embodiments, the pharmaceutical composition comprising the first therapeutic agent is a spray-dried dispersion comprising at least one polymer chosen from Eudragit L100, poly(vinylpyrrolidone), hydroxypropyl methyl cellulose (HPMC), cellulose acetate phthalate (CAP), and hydroxypropyl methylcellulose acetate succinate (HPMCAS-M). In certain embodiments, the pharmaceutical composition comprising the first therapeutic agent is a spray-dried dispersion comprising at least one polymer chosen from Eudragit L100, poly(vinylpyrrolidone) viscosity grade K30 (PVP K30), hydroxypropyl methyl cellulose (HPMC), cellulose acetate phthalate (CAP), and hydroxypropyl methylcellulose acetate succinate (HPMCAS-M). In certain embodiments, the pharmaceutical composition comprising the first therapeutic agent is a spray-dried dispersion comprising at least one polymer chosen from Eudragit L100 and hydroxypropyl methylcellulose acetate succinate (HPMCAS-M). In certain embodiments, the pharmaceutical composition comprising the first therapeutic agent is a spray-dried dispersion comprising Eudragit L100. In certain embodiments, the pharmaceutical composition comprising the first therapeutic agent is a spray-dried dispersion comprising hydroxypropyl methylcellulose acetate succinate (HPMCAS-M).

The formulations of the invention may be buffered by the addition of suitable buffering agents.

In certain embodiments, the oral pharmaceutical composition comprising the first therapeutic agent of the present invention is a unit dose composition. In certain embodiments, the pharmaceutical composition contains about 1 mg to about 5000 mg of the first therapeutic agent. In certain embodiments, the pharmaceutical composition contains about 100 mg to about 3000 mg of the first therapeutic agent. In certain embodiments, the pharmaceutical composition contains about 200 mg to about 2000 mg of the first therapeutic agent. In certain embodiments, the pharmaceutical composition contains about 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2500 mg, or 3000 mg of first therapeutic agent. In certain embodiments, the pharmaceutical composition contains about 300 mg, 500 mg, 700 mg, or 1000 mg of the first therapeutic agent.

In certain embodiments, the pharmaceutical composition of the present invention comprises an emulsion, particle, or gel as described in U.S. Pat. No. 7,220,428. In certain embodiments, the pharmaceutical composition is a solid or liquid formulation having from about 0.1% to about 75% w/w lipids or fatty acid components. In certain embodiments, the formulation contains about 0.1% to about 15% w/v lipids and fatty acid components. In certain embodiments, the fatty acid component comprises saturated or unsaturated C4, C5, C6, C7, C8, C9, C10, C11, or C12 fatty acids and/or salts of such fatty acids. Lipids may include cholesterol and analogs thereof.

The description above describes multiple aspects and embodiments of the invention, including therapeutic methods, pharmaceutical compositions, and medical kits. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and are not intended to limit the invention.

### Example 1—In Vitro Efficacy of 6,8-Bis-benzylthio-octanoic Acid Against Prostate Cancer Cell Lines

The efficacy of 6,8-bis-benzylthio-octanoic acid in killing prostate cancer cell lines was tested in vitro. The tested cell lines represent a diverse set, containing both androgen dependent and independent growth. Experimental procedures and results are described below.

### Part I—Experimental Procedures

A panel of five prostate cancer cell lines was treated with 6,8-bis-benzylthio-octanoic acid in vitro. In particular, prostate cancer cells were treated with 6,8-bis-benzylthio-octanoic acid while in RPMI supplemented with 10% FBS for 60 hrs. Cell survival was assayed with CellTiter 96® Aqueous One Solution Cell Proliferation Assay (Promega). EC₅₀ values were calculated.

### Part II—Results

CPI-613 was highly effective at killing prostate cancer cells in vitro. EC₅₀ values reflecting the ability of 6,8-bis-benzylthio-octanoic acid to kill the indicated prostate cancer cell lines are provided in Table 1 below.

TABLE 1

| Prostate Cancer | EC₅₀ |
|---|---|
| Cell Line | (μM) |
| DU 145 | 99 |
| LnCap | 114 |
| RWPI-1 | 147 |
| 22RV1 | 175 |
| PC-3 | 202 |

### Example 2—In Vitro Efficacy of 6,8-Bis-benzylthio-octanoic Acid in Combination with Docetaxel Against Prostate Cancer Cell Lines

The efficacy of 6,8-bis-benzylthio-octanoic acid in combination with docetaxel to kill prostate cancer cell lines was tested in vitro. Experimental procedures and results are described below.

### Part I—Experimental Procedures

PC-3 androgen resistant prostate cancer cells were treated with subtherapeutic doses of (i) 6,8-bis-benzylthio-octanoic acid, (ii) docetaxel, or (iii) 6,8-bis-benzylthio-octanoic acid and docetaxel. In particular, PC-3 androgen resistant prostate cancer cells were treated for 44 hours in RPMI+10% FBS with (i) a 50 mM solution of 6,8-bis-benzylthio-octanoic acid alone, (ii) either a 2 nM solution of docetaxel or a 200 nM solution of docetaxel alone, or (iii) a combination of 6,8-bis-benzylthio-octanoic acid and docetaxel at the foregoing stated amounts. Prostate cancer cell survival was determined using CellTiterGLO (Promega).

### Part II—Results

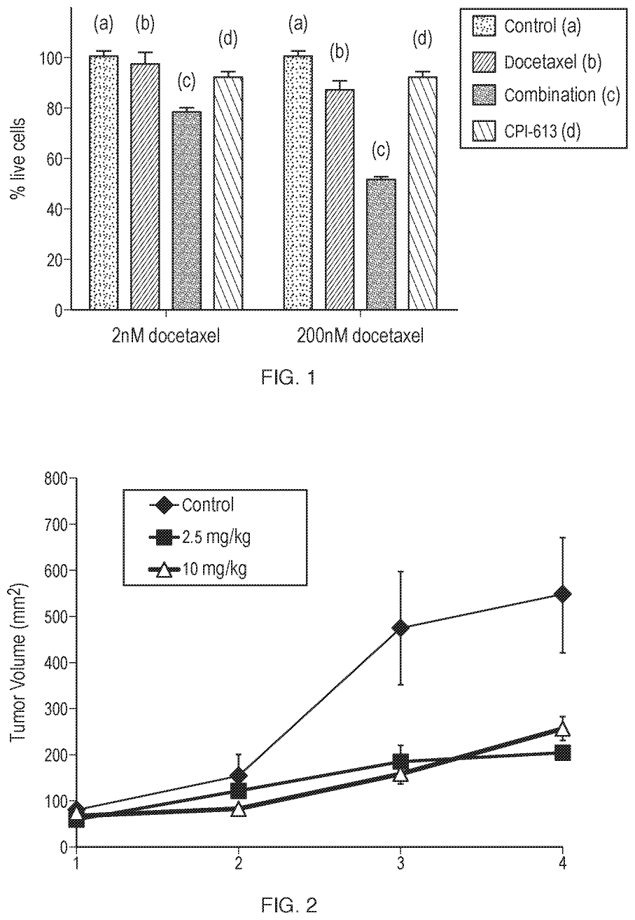

The combination of CPI-613 and docetaxel resulted in significant cell killing compared to each agent alone at subtherapeutic doses, unexpectedly resulting in more cell killing than predicted by adding the levels observed for each agent alone. In other words, we discovered that the combination of CPI-613 and docetaxel is synergistic. The percentage of remaining live PC-3 androgen resistant prostate cancer cells observed following treatment are shown in FIG. 1.

### Example 3—In Vivo Efficacy of 6,8-Bis-benzylthio-octanoic Acid Against Androgen-Resistant Prostate Cancer

The efficacy of 6,8-bis-benzylthio-octanoic acid in killing androgen-resistance prostate cancer cells was tested in vivo in a preclinical PC-3 flank model of androgen-resistant prostate cancer. Experimental procedures and results are described below.

### Part I—Experimental Procedures

Mice were implanted with tumors of androgen-resistance prostate PC-3 cancer cells. After tumors in mice reached approximately 40 mm³, groups of mice (n=8) were treated 2-3 times per week for five weeks with (i) 2.5 mg/kg of 6,8-bis-benzylthio-octanoic acid, (ii) 10 mg/kg of 6,8-bis-benzylthio-octanoic acid, or (iii) saline control. The CPI-613 was formulated in the same manner as it was for clinical trials. Tumors were measured using a caliper. Tumor volume was recorded over four weeks of therapy. Survival of the mice was measured as weeks on therapy in groups of mice treated with 0 mg/kg, 2.5 mg/kg, or 10 mg/kg of 6,8-bis-benzylthio-octanoic acid; experimental endpoints included death, tumor ulceration, or tumor volume reaching 750 mm³.

### Part II—Results

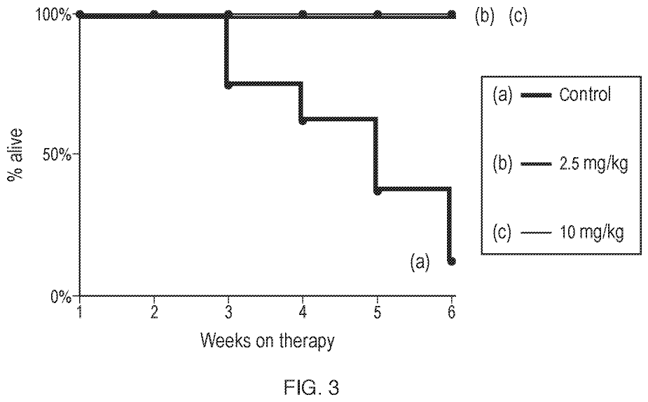

A graph of tumor volume over time is provided in FIG. 2. Tumor growth was significantly slower in groups of mice treated with CPI-613. A graph showing mouse survival time is provided in FIG. 3 (same mice as in FIG. 2). Both groups of mice treated with 6,8-bis-benzylthio-octanoic acid demonstrated a significant decrease in tumor growth and increased survival without toxicity. There was no observed difference between doses. Without wishing to be held to any particular theory, the lack of difference between doses may be due to having crossed the threshold of drug uptake and concentration required in the mitochondria for a response.

### Example 4—Oral Efficacy of 6,8-Bis-benzylthio-octanoic Acid in Non-Small Cell Lung Cancer

Human H460 NSCLC cells were obtained from American Type Cell Culture (ATCC) (catalog no. HTB-177, Manassas, Va.). These cells tested negative for viral contamination using the Mouse Antibody Production (MAP) test, performed by Charles River Labs Molecular Division, upon the receipt of the tumor cells from ATCC. The tumor cells were maintained at 37° C. in a humidified 5% CO₂ atmosphere in T225 tissue culture flasks containing 50 mL of Roswell Park Memorial Institute (RPMI)-1640 solution with 10% Fetal Bovine Serum (FBS) and 2 mM L-glutamine. Cells were split at a ratio of 1:10 every 2-3 days by trypsinization and resuspended in fresh medium in a new flask. Cells were harvested for experiments in the same way at 70-90% confluency.

CD1-Nu/Nu female mice, ˜4-6 weeks old were obtained from Charles River Laboratories. Mice were housed 5 to a cage in a micro-isolator room in the Department of Animal Laboratory Research of New York State University (SUNY) at Stony Brook. Light-dark cycles were 12 h each daily, with light from 7 a.m. to 7 p.m. Food (Purina Rodent Chow) and water (distilled sterile-filtered water, pH 7) were provided ad libitum. Protocols and procedures were according to the rules of and approved by the SUNY Institutional Animal Care and Use Committee (IACUC).

An acclimation period of 7 days was allowed between the arrival of the animal at the study site before tumor inoculation and experimentation. Mice were inoculated subcutaneously (SC) in the right flank with 2×10⁶ human H460 NSCLC or BxPC3 pancreatic cancer cells that were suspended in 0.1 mL of Dulbeco's Phosphate Buffered Salt (PBS) solution using a 1 cc syringe with a 27-5/8 gauge needle. Tumor dimensions (length and width) were measured daily before, during and after treatment (using Vernier calipers) and the tumor volume was calculated using the prolate ellipsoid formula: (length× width²)/2. Treatment with test or control articles began 8 days post tumor cell implantation when the tumor was approximately 300 mm³.

Oral dosing of 6,8-bis-benzylthio-octanoic acid was at 100 mg/kg with 11 animals per group. 100 mg of 6,8-bis-benzylthio-octanoic acid was suspended in a small volume 0.01-0.05N NaOH in 5% dextrose and titrated to pH 7.0 with 4% Glacial Acetic Acid to 50 mg/mL. Prior to administration the suspension was diluted with 5% dextrose to 12.5 mg/mL so that the animals received 100 mg/kg with a dose volume of about 0.2 mL delivered by gastric gavage. Post tumor cell implantation, mice were treated on day 8, day 15, day 22, and day 29.

A similar study was conducted in CD-1 nude mice (n=9) inoculated with 2×10⁶ BxPC-3 cells. The study was initiated when tumors reached an average size of 150 mm³ (day 0) and CPI-613 was administered at an oral dose of 100 mg/week for 4 weeks. A comparator arm (n=9) was conducted with IP treatment at a weekly dose of 25 mg/kg.

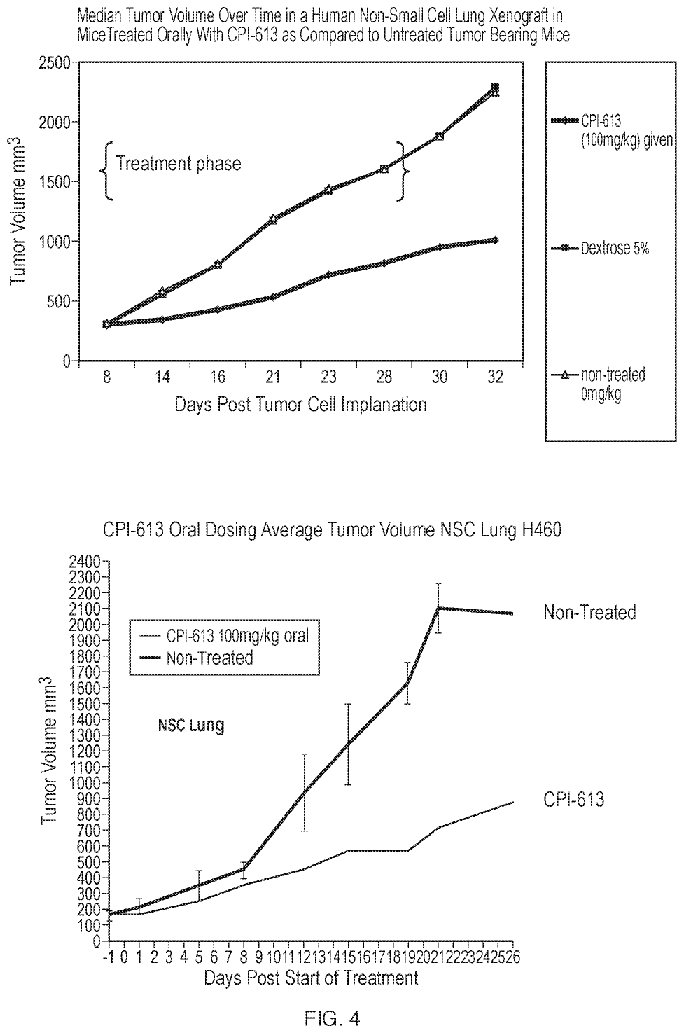

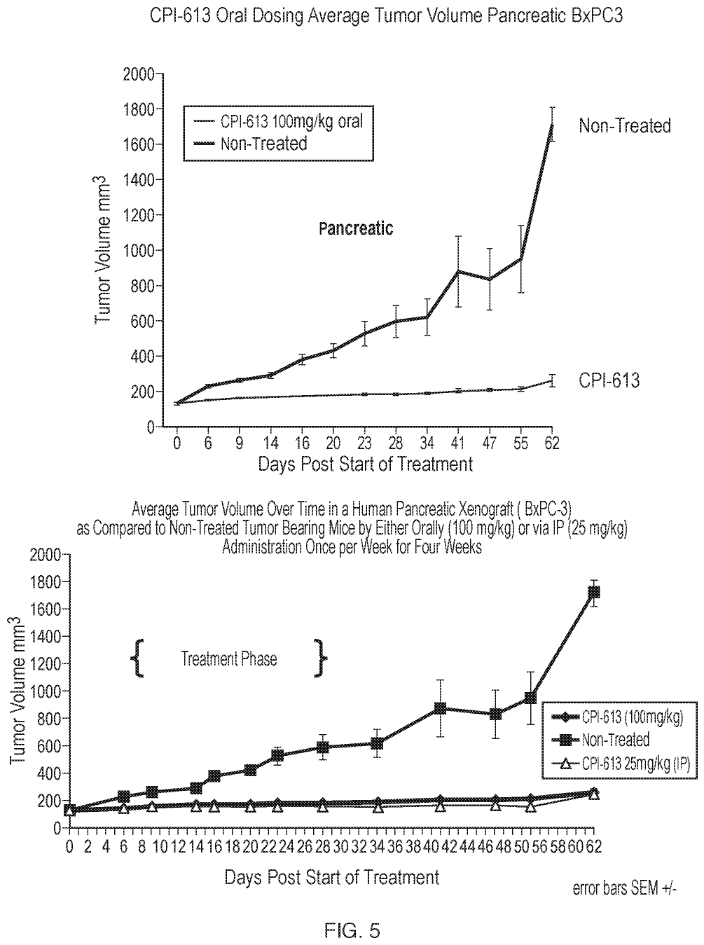

The results are presented in FIGS. 4 and 5. It is evident that the tumors in the mice treated with 6,8-bis-benzylthio-octanoic acid grew much more slowly than those in mice treated with 5% dextrose or untreated. The effect was especially pronounced in BxPC3 tumors. This example demonstrates that 6,8-bis-benzylthio-octanoic acid is effective to treat cancer when administered orally.

### Example 5—Spray Dried Dispersion Oral Formulation of 6,8-Bis-benzylthio-octanoic Acid

Solid amorphous dispersion formulations of 6,8-bis-benzylthio-octanoic acid (API) were prepared by mixing the API 1:4 with one of the following polymers: Eudragit L100, poly(vinylpyrrolidone) viscosity grade K30 (PVP K30), hydroxypropyl methyl cellulose (HPMC), cellulose acetate phthalate (CAP), or hydroxypropyl methylcellulose acetate succinate (HPMCAS-M), and spray drying from methanol or acetone using a small-scale Bend Lab Dryer with 35 kg/hr drying gas flow rate capacity (BLD-35). Conditions, yields, and residual solvent levels of two representative spray dried dispersion (SDD) formulations (75 g each) are presented in the following table.

| Formulation | 20% API:Eudragit L100 | 20% API:HPMCAS-M |
|---|---|---|
| Spray Solution | 5% solids in methanol | 5% solids in acetone |
| Outlet Temp | 45° C. | 35° C. |
| Solution Feed Rate | 35 g/min | |
| Drying Gas Flow Rate | 475-500 g/min | |
| Atomization Pressure | 120 psi | |
| Nozzle | Schtick 2.0 pressure swirl atomizer | |
| Secondary Drying | 20 hr at 30° C. | |
| Dry Yield (%) | 94 | 96 |
| Residual Solvent (%) | 4.21 ± 0.02 | 1.01 ± 0.00 |
| (Wet SDD) | (MeOH) | (Acetone) |
| Residual Solvent (%) | <LOQ | <LOQ |
| (Tray-Dried Material) | | |
| API content by HPLC | 201 ± 1.1 mg/g | 198 ± 0.2 mg/g |

Scanning electron microscopy (SEM) was used to qualitatively determine particle morphology of the two SDD formulations, and to study if any degree of fusion or crystallinity was visually present. Particles show collapsed sphere morphology with no crystallization or fusion noted.

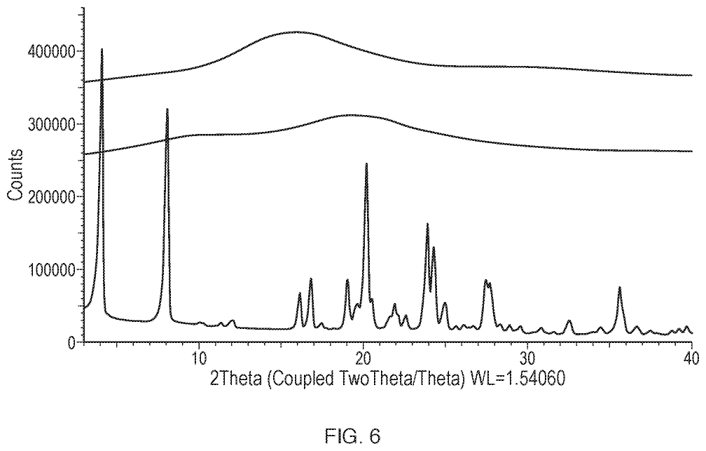

X-ray diffraction is typically sensitive to the presence of crystalline material with an LOD of about 1% of the sample mass. No crystallinity was detected by PXRD for either SDD formulation. Diffractograms in comparison to crystalline 6,8-bis-benzylthio-octanoic acid API can be found in FIG. 6, wherein the top diffractogram is the Eudragit L100 formulation, the middle diffractogram is the HPMCAS-M formulation, and the bottom diffractogram is crystalline 6,8-bis-benzylthio-octanoic acid.

### Example 6—Emulsion Oral Formulations of 6,8-Bis-benzylthio-octanoic Acid

Monolaurin (131 mg) and 6,8-bis-benzylthio-octanoic acid (93 mg) were warmed to 50° C. in polysorbate-80 (2.5 mL) in a round bottomed flask equipped with a magnetic stir bar. After complete dissolution to a clear solution, water (7.5 mL) was added with vigorous stirring at 50° C. to provide an emulsion.

6,8-bis-benzylthio-octanoic acid (312 mg) was combined with polysorbate 80 (6.25 g), soybean oil (1.25 g), and a lipid mix (100 mg) comprising cholesterol (14 g), cholesteryl acetate (14 g), cholesteryl benzoate (14 g), monolaurin (25.4 g), and monopalmitin (32.6 g), and the mixture heated to 50° C. until the solids dissolved (30 min). Dextrose (11.25 g) was dissolved in 236 mL of water, and the resulting aqueous dextrose solution was added to the oil solution above. The resulting two phase mixture was stirred for 30 min at rt, then vacuum filtered through a 0.22 um filter.

### Example 7—Liquid Formulations of 6,8-Bis-benzylthio-octanoic Acid

A 6,8-bis-benzylthio-octanoic acid solution was prepared by the steps of (a) providing a 50 mg/mL solution of 6,8-bis-benzylthio-octanoic acid in 1 M aqueous triethanolamine, and (b) diluting the 50 mg/mL solution with 5% aqueous dextrose to a concentration of 5 mg/mL. The resulting 5 mg/mL solution is identified as “7A” in Example 8 below.

A suspension vehicle was prepared by the steps of: (a) combining tris buffer (48 mg) and HPMCAS-HF (20 mg) in 14 mL of distilled water, (b) adjusting the pH to 7.4 with dilute sodium hydroxide to dissolve the HPMCAS-HF, (c) heating the resulting solution to approximately 90° C., (d) adding Methocel A4M Premium (100 mg) to the hot solution, (e) stirring the mixture vigorously to suspend the undissolved Methocel A4M, (0 cooling and stirring the mixture with an ice bath until the Methocel A4M dissolves (approximately 10 minutes), (g) diluting the solution with distilled/deionized water to bring the total volume to 20 mL, and (h) adjusting the pH to 7.4 with dilute acetic acid or dilute sodium hydroxide to provide the suspension vehicle.

Suspensions of the spray-dried formulations of Example 5 were prepared by adding 400 mg of the respective SDD formulation to a mortar, slowly adding 4 mL of the suspension vehicle (mixing thoroughly with a pestle after each small addition to uniformly disperse), and then transferring to a flask and stirring for one minute prior to administration. The resulting suspension of the Eudragit L100 SDD formulation (20 mg/mL 6,8-bis-benzylthio-octanoic acid) is identified as “7B” in Example 8 below. The resulting suspension of the HPMCAS-M SDD formulation (20 mg/mL 6,8-bis-benzylthio-octanoic acid) is identified as “7C” in Example 8 below.

In the same way, a 20 mg/mL suspension of 6,8-bis-benzylthio-octanoic acid was prepared by adding 80 mg 6,8-bis-benzylthio-octanoic acid to a mortar, slowly adding 4 mL of the suspension vehicle (mixing thoroughly with a pestle after each small addition to uniformly disperse), and then transferring to a flask and stirring for one minute prior to administration. The resulting suspension of 6,8-bis-benzylthio-octanoic acid is identified as “7D” in Example 8 below.

A solution of 6,8-bis-benzylthio-octanoic acid was prepared by dissolving SOLUTOL® (polyoxyl 15 hydroxystearate; KOLLIPHOR® HS 15) (3 grams) in distilled water (7 mL) to form a 30% solution, adding 6,8-bis-benzylthio-octanoic acid (50 mg) to 5 mL of the 30% solution, vortexing for 1 minute, and then sonicating for 45 minutes to provide a clear colorless solution (10 mg/mL; pH 7). The resulting solution is identified as “7E” in Example 8 below.

### Example 8—Oral Bioavailability of 6,8-Bis-benzylthio-octanoic Acid

Six groups of 16 BALB/c nude mice (8 males and 8 females) per group were administered 6,8-bis-benzylthio-octanoic acid in six different ways: (1) 5 μL/g IV injection (tail vein) of the triethanolamine/dextrose aqueous solution of Example 7 (25 mg/kg; 5 mL/kg; Ex. 7A); (2) 5 μL/g IP injection of the triethanolamine/dextrose aqueous solution of Example 7 (25 mg/kg; 5 mL/kg; 7A); (3) 5 μL/g oral administration of the Eudragit L100 SDD suspension of Example 7 (100 mg/kg; 5 mL/kg; 7B); (4) 5 μL/g oral administration of the HPMCAS-M SDD suspension of Example 7 (100 mg/kg; 5 mL/kg; 7C); (5) 5 μL/g oral administration of the 20 mg/mL 6,8-bis-benzylthio-octanoic acid suspension of Example 7 (100 mg/kg; 5 mL/kg; 7D); or (6) 10 μL/g oral administration of the 10 mg/mL SOLUTOL solution of Example 7 (100 mg/kg; 10 mL/kg; 7E). In each experiment, about 80 μL of blood was collected from one subgroup of 4 male and 4 female mice at 0.083, 1, 4, and 24 hours after dosing, and from the other subgroup of 4 male and 4 female mice at 0.5, 2, and 8 hours. Plasma from the collected blood samples was analyzed by LC-MS/MS for the presence of 6,8-bis-benzylthio-octanoic acid.

| | | | | | AUC | | | |
|---|---|---|---|---|---|---|---|---|
| | | Mice | Dose | Bioavailability | Last | Cmax | Tmax | T½ |
| Formulation | Route | (n) | (mg/kg) | (%) | (uM*hr) | (uM) | (hr) | (hr) |
| 7A (TEA/dextrose) | IV | 16 | 25 | — | 36 | 92 | 0.08 | 2.0 |
| 7A (TEA/dextrose) | IP | 16 | 25 | 83 | 29 | 103 | 0.08 | 3.9 |
| 7B (Eudragit SDD) | PO | 16 | 100 | 44 | 61 | 94 | 0.08 | 2.0 |
| 7C (HPMCAS-M SDD) | PO | 16 | 100 | 43 | 60 | 69 | 0.08 | 1.1 |
| 7D (CPI-613) | PO | 16 | 100 | 57 | 82 | 82 | 0.50 | 3.7 |
| 7E (Solutol) | PO | 16 | 100 | 127 | 175 | 229 | 0.08 | 4.4 |

This example demonstrates that 6,8-bis-benzylthio-octanoic acid is orally bioavailable.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for treating a cancer selected from the group consisting of androgen-dependent prostate cancer and androgen-independent prostate cancer, comprising administering to a patient in need thereof a therapeutically effective amount of a first therapeutic agent comprising 6,8-bis-benzylthio-octanoic acid or a pharmaceutically acceptable salt thereof, in order to treat the cancer.

2. The method of claim 1, wherein the cancer is androgen-dependent prostate cancer.

3. The method of claim 1, wherein the cancer is androgen-independent prostate cancer.

4. The method of claim 1, wherein the cancer is metastatic.

5. The method of any one of claims 1-4, further comprising administering to the patient a second therapeutic agent having anti-cancer activity.

6. A method for treating prostate cancer, comprising administering to a patient in need thereof a therapeutically effective amount of (i) a first therapeutic agent comprising 6,8-bis-benzylthio-octanoic acid or a pharmaceutically acceptable salt thereof and (ii) a second therapeutic agent comprising docetaxel or a pharmaceutically acceptable salt thereof, in order to treat the prostate cancer.

7. The method of claim 6, wherein the prostate cancer is androgen-dependent prostate cancer.

8. The method of claim 6, wherein the prostate cancer is androgen-independent prostate cancer.

9. The method of claim 6, wherein the prostate cancer is metastatic.

10. The method of any one of claims 6-9, wherein the second therapeutic agent is docetaxel.

11. The method of any one of claims 6-9, wherein the second therapeutic agent is administered at a dosage ranging from about 50 mg/m² to about 100 mg/m².

12. The method of any one of claims 6-9, wherein the second therapeutic agent is administered at a dosage of about 75 mg/m².

13. The method of any one of claims 6-12, wherein the second therapeutic agent is administered once per any three week period.

14. The method of any one of claims 6-13, wherein the second therapeutic agent is administered by intravenous administration.

15. The method of any one of claims 1-14, wherein the first therapeutic agent is administered at a unit dosage ranging from about 150 mg/m² to about 1500 mg/m².

16. The method of any one of claims 1-14, wherein the first therapeutic agent is administered at a unit dosage ranging from about 500 mg/m² to about 1000 mg/m².

17. The method of any one of claims 1-16, wherein the first therapeutic agent is administered orally.

18. The method of any one of claims 1-16, wherein the first therapeutic agent is administered by intravenous administration.

19. The method of any one of claims 1-18, wherein the first therapeutic agent is administered to the patient no more frequently than once per day.

20. The method of any one of claims 1-18, wherein the first therapeutic agent is administered to the patient twice per week for at least two weeks.

21. The method of any one of claims 1-18, wherein the first therapeutic agent is administered to the patient once every two weeks.

22. A method for treating prostate cancer, comprising administering to a patient in need thereof a unit dosage of a first therapeutic agent comprising 6,8-bis-benzylthio-octanoic acid or a pharmaceutically acceptable salt thereof in an amount ranging from about 150 mg/m² to about 1500 mg/m², to treat the prostate cancer.

23. The method of claim 22, wherein the unit dosage is from about 500 mg/m² to about 1000 mg/m² of 6,8-bis-benzylthio-octanoic acid or a pharmaceutically acceptable salt thereof.

24. The method of claim 22 or 23, wherein the unit dosage is administered to the patient no more frequently than once per day.

25. The method of claim 22 or 23, wherein the unit dosage is administered to the patient twice per week.

26. The method of claim 22 or 23, wherein the unit dosage is administered to the patient once every two weeks.

27. The method of any one of claims 22-26, wherein the unit dosage is administered intravenously to the patient.

28. The method of any one of claims 22-26, wherein the unit dosage is administered orally to the patient.

29. The method of any one of claims 1-28, wherein the first therapeutic agent is administered in the form of a pharmaceutical composition comprising 6,8-bis-benzylthio-octanoic acid and a pharmaceutically acceptable carrier.

30. The method of any one of claims 1-28, wherein the first therapeutic agent is administered in the form of a pharmaceutical composition comprising 6,8-bis-benzylthio-octanoic acid and an ion pairing agent.

31. The method of any one of claims 1-28, wherein the first therapeutic agent is administered in the form of a pharmaceutical composition comprising 6,8-bis-benzylthio-octanoic acid and triethanolamine.

32. The method of any one of claims 1-31, wherein the patient is an adult human.

33. The method of any one of claims 1-32, wherein the patient is at least partially refractory to docetaxel.

34. A medical kit, comprising (i) a first therapeutic agent comprising 6,8-bis-benzylthio-octanoic acid or a pharmaceutically acceptable salt thereof, and (ii) instructions for treating a cancer selected from the group consisting of androgen-dependent prostate cancer and androgen-independent prostate cancer using said first therapeutic agent.

35. A medical kit, comprising (i) a first therapeutic agent comprising 6,8-bis-benzylthio-octanoic acid or a pharmaceutically acceptable salt thereof, and (ii) instructions for treating prostate cancer using the first therapeutic agent in combination with a second therapeutic agent comprising docetaxel or a pharmaceutically acceptable salt thereof.

36. A medical kit, comprising (i) a first therapeutic agent comprising 6,8-bis-benzylthio-octanoic acid or a pharmaceutically acceptable salt thereof, and (ii) instructions for treating prostate cancer using a dosage of the first therapeutic agent ranging from about 150 mg/m² to about 1500 mg/m².
